(19) United States
(12) Patent Application Publication
(10) Pub. No.: US 2017/0296240 A1
(43) Pub. Date: Oct. 19, 2017

(54) SPINAL FIXATION AIMING DEVICE

(71) Applicant: Shao-Kang Hsueh, New Taipei City (TW)

(72) Inventor: Shao-Kang Hsueh, New Taipei City (TW)

(21) Appl. No.: 15/098,319

(22) Filed: Apr. 14, 2016

(51) Int. Cl. A61B 17/70; A61B 90/50

(52) U.S. Cl. CPC A61B 17/7083; A61B 90/50

(57) ABSTRACT

A spinal fixation aiming device including an arc guide rail; an arc slide unit movably coupled to the arc guide rail; and a tool holder mounted to the arc slide unit and movable to a predetermined position to be selectively fixed at the position. To operate, the arc guide rail is fixed to an operation table and the arc slide unit and the tool holder are adjusted for desired angles for a surgical operation. The adjustment is conducted such that through collaborative combination between the arc guide rail and the arc slide unit, the tool holder is constantly kept corresponding to a spinal fixation nailing site of a patient so as to achieve an effect of easy operation and accurate aiming

### TECHNICAL FIELD OF THE INVENTION

The present invention relates generally to a novel spinal fixation aiming device, and more particularly to one for use in spine related surgeries.

### DESCRIPTION OF THE PRIOR ART

The modern spinal fixation surgeries are generally conducted by incising the back of a patient along a central line and cutting off supraspinous ligaments. The muscles next to the spine are pushed aside by periosteal elevators to a nailing site for fixation and the muscles are held in such a position by being pulled to two sides by hooks. This leads to tendons that are attached to the spine being cut off and damage of the muscles and tendons around the spine would inevitably occur. The following disadvantage would happen:

(1) When the surgery is completed and the muscles are stitched back, the muscles and tendons that are around the spine cannot be placed back and held at the site before the surgery and this would severely damage the muscles of the surgery patient.

(2) There is an excessively wide dead space and this would lead to epidural fibrosis after the surgery, making the result of operation poor.

(3) The spine would become unstable after the surgery due to supraspinous ligaments, vertebral spine, and interspinous ligaments of the patient all being cut off

To overcome the drawbacks of the conventional spinal fixation surgeries, minimal invasion spinal fixation surgeries that have a minimal incision and the minimum extent of damage of muscular tissues have been proposed. Two commonly used processes have been adopted for minimal invasion spinal fixation surgeries. The first one is that fixation is conducted by way of percutaneous pedicle and implantation and the second one is using an expansion sleeve to conduct fixation with visual observation. However, there are still drawbacks as follows:

(I) Fixation by Way of Percutaneous Pedicle:

(1) Excessive light transmission would occur during the process of the surgery and may cause damage to the vision of the surgeons and the patient.

(2) For most bone fractures of high location lumbar vertebra or thoracic vertebra, retrogressive low waist detachment or instability of spine would result, leading to high difficulty of surgery and being not used often.

(3) The implants and devices used in the surgery are specially designed for percutaneous surgeries and thus, the cost for developing such surgery devices is expensive.

(II) Fixation by Way of Expansion Sleeve:

(1) Although direct visual observation is possible for surgeons, the fixation nailing site is often full of densely distributed blood vessels related to wide circulation, making it often to cause massive bleeding during the operation. In addition, the surgery wound at the nailing site is generally deep, leading to undesired influence on the eyesight of the surgeon and thus difficulty of the surgical operation.

(2) The expansion sleeve, the implant, and the surgery devices fused in the surgical operation may often hit each other and thus cause interference with the performance of the surgical operation.

(3) The expansion sleeve used in the surgery is not light transmittable and may block the light of a surgical lighting device during the performance of the surgery.

### SUMMARY OF THE INVENTION

The primary object of the present invention is to provide a spinal fixation aiming device, which comprises an arc guide rail, which when put into use, is fixed to an operation table and an arc slide unit and a tool holder are movable are adjusted for desired angles for a surgical operation. The adjustment is conducted such that through collaborative combination between the arc guide rail and the arc slide unit, the tool holder is constantly kept corresponding to a spinal fixation nailing site of a patient so as to achieve an effect of easy operation and accurate aiming

To achieve the above object, the present invention provides a spinal fixation aiming device, which comprises: an arc guide rail; an arc slide unit movably coupled to the arc guide rail; and a tool holder mounted to the arc slide unit and movable to a predetermined position to be selectively fixed at the position.

In an embodiment of the present invention, the arc guide rail has an end to which a fixing bar is mounted.

In an embodiment of the present invention, the arc slide unit comprises a base, guide sections respectively extending from opposite ends of the base, and through holes respectively formed in the guide sections for movably coupling to the arc guide rail.

In an embodiment of the present invention, the tool holder comprises a tool support section, slidable sleeves respectively mounted to opposite sides of the tool support section and respectively coupled to the guide sections, internal threading sections respectively extending to and communicating with the slidable sleeves, and threading fasteners respectively engaging with the internal threading sections.

In an embodiment of the present invention, the arc guide rail, the arc slide unit, and the tool holder are each made of a light-transmitting material.

The foregoing objectives and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 is a perspective view of the present invention.

FIG. 2 is an exploded view of the present invention.

FIG. 3 is a cross-sectional view illustrating an operation condition of the present invention.

### DETAILED DESCRIPTION OF THE PREFERRED EMBODIMENTS

The following descriptions are exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

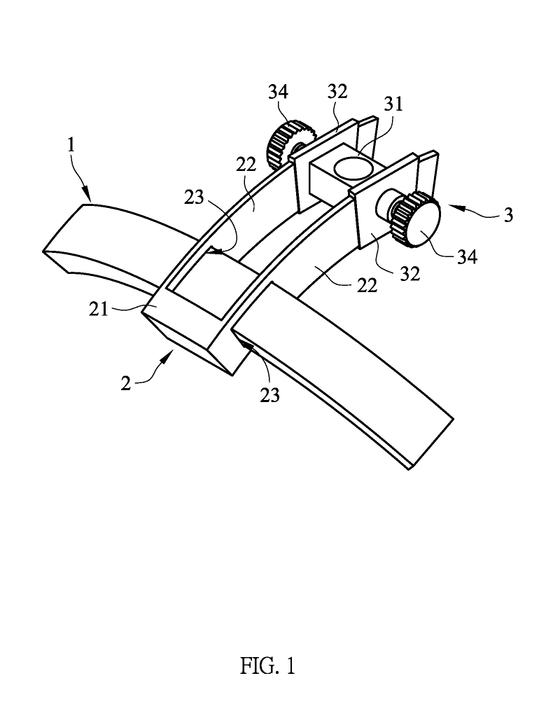

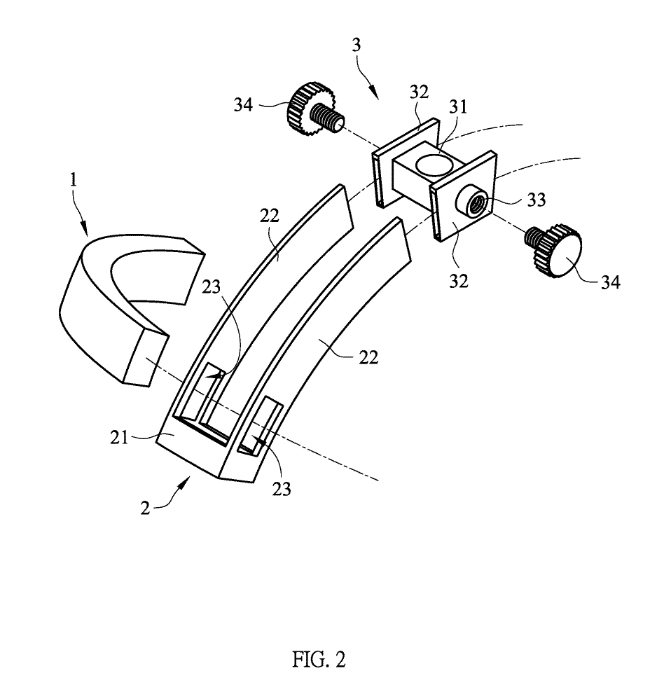

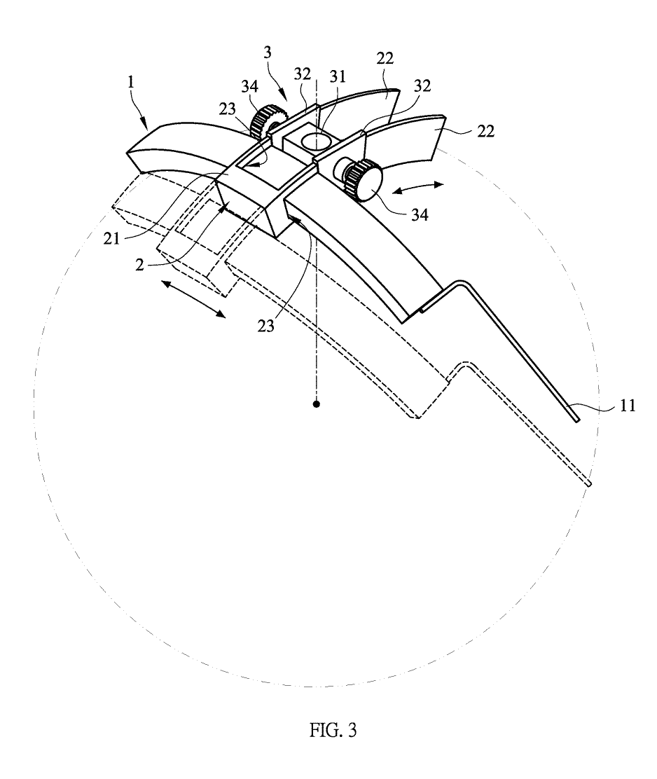

Referring to FIGS. 1-3, which are respectively a perspective view and an exploded view of the present invention and a cross-sectional view illustrating an operation of the present invention, as shown in the drawings, the present invention provides a spinal fixation aiming device, which comprises at least an arc guide rail 1, an arc slide unit 2, and a tool holder 3. The arc guide rail 1, the arc slide unit 2, and the tool holder 3 are each made of a light-transmitting material.

The arc guide rail 1 has an end to which a fixing bar 11 is mounted.

The arc slide unit 2 is movably coupled to the arc guide rail 1. The arc slide unit 2 comprises a base 21, guide sections 22 respectively extending from opposite ends of the base 21 and opposite to each other, and through holes 23 respectively formed in the guide sections 22 for movably coupling to the arc guide rail 1.

The tool holder 3 is mounted to the arc slide unit 2 and is movable to a predetermined location and selectively fixed at the location. The tool holder 3 comprises a tool support section 31, slidable sleeves 32 respectively mounted to opposite sides of the tool support section 31 and respectively coupled to the guide sections 22, internal threading sections 33 respectively extending to and communicating with the slidable sleeves 32, and threading fasteners 34 respectively engaging with the internal threading sections 33. As such, with the above-described technical features properly assembled, a novel spinal fixation aiming device is provided.

To use the present invention, the arc guide rail 1 is fixed by the fixing bar 11 to an operation table (not shown) to allow for adjustment of the angle and position of the arc slide unit 2 on the arc guide rail 1. To conduct the adjustment, a surgeon may directly apply a force to the base 21 (or one or two of the guide sections 22) for movement thereof, whereby the guide sections 22 are moved by means of the through holes 23 sliding along the arc guide rail 1 in order to adjust the angle thereof, in a left-right direction, (the movable range of angle being for example 0-45 degrees), and then a desired position of the tool holder 3 on the arc slide unit 2 is adjusted. To adjust the position, the surgeon may directly apply a force to one of the threading fasteners 34 to cause the slidable sleeves 32 to move along the guide sections 22 in order to adjust the angle thereof, in a front-rear direction, (the movable range of angle being for example 0-45 degrees). After the desired position is reached through the adjustment, a force is applied to screw each of the threading fasteners 34 into the respective internal threading section 33 in such a way that an end of each of the threading fasteners 34 is made abutting the respective guide sections 22 for fixing. Further, the arc guide rail 1 and the arc slide unit 2 are arranged together to form a spherical surface, and the tool support section 31 is kept to always aim at a spinal fixation nailing site of the patient (which corresponds to the center of the sphere) for any position to which the arc slide unit 2 or the tool holder 3 is moved so that a surgical device can be easily mounted to and supported on the tool support section 31 to allow the surgeons to easily carry out the surgical operation.

Further, the arc guide rail 1, the arc slide unit 2, and the tool holder 3 are designed to be formed of light-transmitting materials so that no shadow may be cast by the lighting of surgical lights, allowing for a better range of eyesight of the surgeon during the surgical operation. Further, after the adjustment has been done and the nailing site adjacent to the spine of the patient has been fixed by the surgical device, X-ray photographing may be conducted directly so that the time period in which the surgeon may be exposed to X-ray radiation is significantly shortened and convenience of use during the surgical operation may be achieved.

In summary, the present invention provides a spinal fixation aiming device that effectively overcomes the drawbacks and shortcomings of the prior art devices and allows an arc guide rail to be fixed to an operation table during the use thereof and allows for adjustment of angles of an arc slide unit and a tool holder to suit the needs of the surgical operation. During the adjustment conducted by a surgeon, the collaborative combination between the arc guide rail and the arc slide unit allows the tool holder to aim at or correspond to the spinal fixation nailing site of the patient at any angle so as to achieve an effect of easy operation and accurate aiming

It will be understood that each of the elements described above, or two or more together may also find a useful application in other types of methods differing from the type described above.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the claims of the present invention.

1. A spinal fixation aiming device, comprising:
an arc guide rail, which defines a first spherical arc;
an arc slide unit, which is movably coupled to the arc guide rail to be selectively movable along the first spherical arc and defines a second spherical arc that is different from the first spherical arc; and
a tool holder, which is mounted to the arc slide unit and is movable along the second spherical arc defined by the arc slide unit to a predetermined position and selectively fixed at the position
wherein the first spherical arc and the second spherical arc are different from each other and collectively define a spherical surface having a center that the tool holder aims at so that the tool holder is movable on the spherical surface.

2. The spinal fixation aiming device according to claim 1, wherein the arc guide rail has an end to which a fixing bar is mounted.

3. The spinal fixation aiming device according to claim 2, wherein the arc slide unit comprises a base, guide sections respectively extending from opposite ends of the base, and through holes respectively formed in the guide sections for movably coupling to the arc guide rail.

4. The spinal fixation aiming device according to claim 3, wherein the tool holder comprises a tool support section, slidable sleeves respectively mounted to opposite sides of the tool support section and respectively coupled to the guide sections, internal threading sections respectively extending to and communicating with the slidable sleeves, and threading fasteners respectively engaging with the internal threading sections.

5. The spinal fixation aiming device according to claim 4, wherein the arc guide rail, the arc slide unit, and the tool holder are each made of a light-transmitting material.
